(19) United States
(12) Patent Application Publication
(10) Pub. No.: US 2020/0383926 A1
(43) Pub. Date: Dec. 10, 2020

(54) ENTERIC COATED PHARMACEUTICAL COMPOSITION AND USE THEREOF

(71) Applicants: SHIN ERA TECHNOLOGY CO. LTD., Taipei City (TW); G2 INNOVATIVE MEDHEALTH INC., Princeton, NJ (US)

(72) Inventors: Chi-Ming Chiu, Taipei City (TW); Ming-Shuen Hong, Changhua City (TW); Po-Wei Yang, Taipei City (TW); Gau-Min DUH, Taipei City (TW); Ming-Houng CHIU, Taipei City (TW)

(21) Appl. No.: 16/767,568

(22) PCT Filed: Nov. 29, 2018
(86) PCT No.: PCT/CN2018/118076

§ 371 Date: May 27, 2020

Related U.S. Application Data
(60) Provisional application No. 62/592,259, filed on Nov. 29, 2017.

(51) Int. Cl. A61K 9/28; A61K 9/20; A61K 9/48; A61K 9/50; A61K 31/722

(52) U.S. Cl. CPC A61K 9/2846; A61K 9/2013; A61K 9/2866; A61K 9/4858; A61K 31/722; A61K 9/5015; A61K 9/5026; A61K 9/5042; A61K 9/4866

(57) ABSTRACT

Provided are an enteric coated pharmaceutical composition and use thereof. The enteric coated pharmaceutical composition comprises chitosan citrate, a pharmaceutically acceptable carrier, and an enteric coating, wherein chitosan citrate is coated by the enteric coating and comprises a chitosan moiety of 50 to 75% deacetylation.

### TECHNICAL FIELD

The present invention relates to a phosphate-binding agent for hyperphosphatemia patients, especially for an enteric coated pharmaceutical composition containing phosphate-binding agent for hyperphosphatemia patients.

### DESCRIPTION OF RELATED ART

Hyperphosphatemia is a syndrome that the serum phosphate level of a subject is above a certain threshold causing deposits throughout the body and endangering circulation. There are many different disease may cause hyperphosphatemia, such as, renal disease, kidney failure, hypoparathyroidism, etc. In end-stage of the aforesaid diseases, the patient's kidney function will be compromised and thereby the phosphate level in the blood becomes markedly elevated.

Patients suffering hypoparathyroidism are advised to have low phosphate diet. However, phosphate is very commonly present in food we eat everyday. That said, it is difficult to avoid foods containing phosphate. Since it is nearly impossible to eat only food containing low phosphate, others strategies shall be adopted for decreasing phosphate uptake. In this regard, phosphate binders are developed for this purpose. Typically, phosphate binders are substances taken orally and effective in the intestinal tract for binding with phosphate and preventing the same from being uptaken.

Conventional phosphate binders include, for example, various salts of aluminum and calcium, as well as some chemically synthesized cross-linked polymers. However, aluminum or calcium salts and cross-linked polymers may both cause undesirable results and therefore, are not ideal.

To sum up, although there are several medicines being used as phosphate binders exist, it is still continuous needs in the art for more options capable of relieving hyperphosphatemia symptom.

### SUMMARY

One of the objectives of the present invention is to provide an alternative phosphate-binding agent that may exhibit better phosphate-binding ability and/or fewer side effects than the conventional medicine used clinically.

Another objective of the present invention is to provide a novel method for treating hyperphosphatemia by using a novel phosphate-binding agent.

In order to achieve the aforesaid objectives, the present invention provides a method for phosphate-binding comprising administering a subject in need an effective amount of an enteric coated pharmaceutical composition comprising chitosan citrate and a pharmaceutically acceptable carrier; wherein said chitosan citrate comprises a chitosan moiety of 50 to 75% deacetylation. Said method can be practiced in vivo, ex vivo, or in vitro.

The present invention also provides an enteric coated pharmaceutical composition, comprising chitosan citrate, a pharmaceutically acceptable carrier, and an enteric coating; wherein said chitosan citrate is coated by said enteric coating; wherein said chitosan citrate comprises a chitosan moiety of 50 to 75% deacetylation.

Preferably, said chitosan moiety is of 55 to 70% deacetylation; more preferably, 57 to 65%; even more preferably, 60 to 63%.

Preferably, said pharmaceutically acceptable carrier comprises water, PBS, salt solutions, gelatins, oils, alcohols, glycerol, chitosan, alginate, chondroitin, Vitamin E, dimethyl sulfoxide (DMSO), or a combination thereof.

Preferably, said subject in need is a hyperphosphatemia patient.

Preferably, said enteric coated pharmaceutical composition is a tablet, a capsule or a microcapsule. Preferably, said administering is through oral route.

Preferably, said administering is conducted before, simultaneously, or after diet of said subject in need.

Preferably, said enteric coated pharmaceutical composition comprises an enteric coating of methacrylic acid and acid ester polymers, polyvinyl acetate phthalate, cellacephate, cellulose acetate trimellitrate, carboxymethyl ethylcellulose, hydroxypropyl methylcellulose phthalate, hydroxypropyl methylcellulose acetate succinate, or a combination thereof.

Preferably, said enteric coated pharmaceutical composition further comprise a pharmaceutically acceptable excipient. Preferably, said excipient comprises a disintegrating agent, a binder, a lubricant, a preservative, or a combination thereof.

Preferably, said enteric coating comprises methacrylic acid and acid ester polymers, polyvinyl acetate phthalate, cellacephate, cellulose acetate trimellitrate, carboxymethyl ethylcellulose, hydroxypropyl methylcellulose phthalate, hydroxypropyl methylcellulose acetate succinate, or a combination thereof.

### BRIEF DESCRIPTION OF THE DRAWINGS

FIG. 1 shows the flow chart of preparing the chitosan citrate of the present invention.

### DETAILED DESCRIPTION

The term “chitosan citrate” is referred to as a conjugate comprising a chitosan moiety and a citric acid moiety. Said chitosan moiety and said citric acid moiety are conjugated together preferably through the hydroxyl groups of said chitosan moiety. In a preferable embodiment, said chitosan moiety is of 50% to 75% deacetylation. In a more preferable embodiment, said chitosan moiety is of 55% to 70% deacetylation. In another preferable embodiment, the deacetylation of said chitosan moiety is 55%, 56%, 57%, 58%, 59%, 60%, 61%, 62%, 63%, 64%, 65%, 66%, 67%, 68%, 69%, or 70%. In an alternative embodiment, said chitosan moiety has a weight-average molecular weight of 15000 to 40000 dalton; preferably, said weight-average molecular weight is of 20000 to 35000 dalton.

It is a common knowledge that the more deacetylation the more active functional groups for phosphate binding and thereby contributes better phosphate-binding ability. Thus, the chitosan-based phosphate-binding agent in the field usually used chitosan of deacetylation at least 90%. However, the research of the present invention surprisedly noted that chitosan of lower deacetylation (50 to 75%) provides better phosphate-binding ability than higher ones (at least 90%).

Without being bound by theory, the present invention proposed that chitosan of lower deacetylation exhibits better phosphate-binding ability in weak-base environment (about pH 8˜9). Since the phosphate uptake in human body actually takes place in the intestine instead of stomach, practically, chitosan of lower deacetylation would have better efficacy in clinical application. The research of the present invention conquered the technical bias existing in the field.

The terms “enteric coated pharmaceutical composition” is referred to a pharmaceutical composition formulated with an enteric coating in order to release the active ingredient in the intestine instead of stomach. In an alternative embodiment, said enteric coating have a dissociation constant (pKa) to ensure that it stays intact in an acidic environment but dissociates and therefore releasing said chitosan citrate in an alkaline environment. In an exemplary embodiment, said enteric coating includes but not limits to methacrylic acid and acid ester polymers, polyvinyl acetate phthalate, cellacephate, cellulose acetate trimellitrate, carboxymethyl ethylcellulose, hydroxypropyl methylcellulose phthalate, and hydroxypropyl methylcellulose acetate succinate.

The term “pharmaceutically acceptable” means non-toxic to the subject and having no interfere with the efficacy of the active ingredient of the pharmaceutical composition at issue. Said pharmaceutically acceptable carrier includes but not limit to water, PBS, salt solutions, gelatins, oils, alcohols, glycerol, chitosan, alginate, chondroitin, Vitamin E, dimethyl sulfoxide (DMSO) or a combination thereof.

The term “an effective amount” or “a therapeutically effective amount” used herein is referred to the amount of each active agent required to confer the desired effect (ex. binding phosphate of the diet in the intestine) on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In an alternative embodiment, said enteric coated pharmaceutical composition can be formulated as a tablet, a capsule or microcapsule. Preferably, said enteric coated pharmaceutical composition is administered orally. In an alternative embodiment, said enteric coated pharmaceutical composition is administered before, simultaneously, or after diet of said subject in need. In an alternative embodiment, said enteric coated pharmaceutical composition further comprises a pharmaceutically acceptable excipient. Said excipient includes but not limits to a disintegrating agent, a binder, a lubricant, a preservative, or a combination thereof.

### Experiment 1: Preparation of the Present Chitosan Citrate

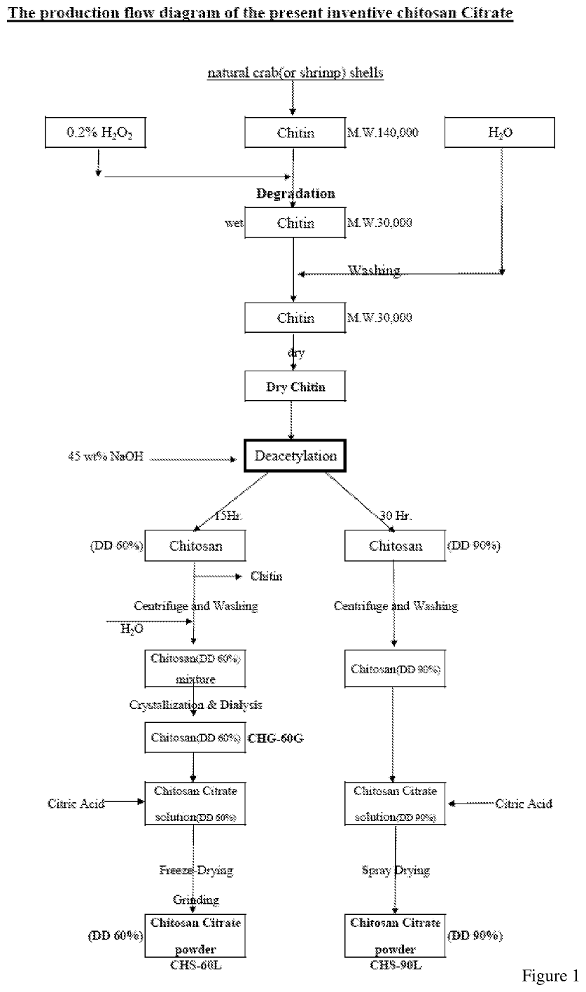

In this example, an exemplary procedure of the present invention was exhibited for preparing the chitosan citrate of the present invention. The preparation was conducted through the following steps and described in FIG. 1:

(1) Chitin extracted from crab (or shrimp) shells whose molecular weight of more than 140,000 dalton was obtained and degraded by hydrogen peroxide (0.2 wt %) at 20° C. for 72 hours. After the degradation, the weight-average molecular weight (according to standard measurement of CHS-E11-03 of Shin Era Technology Co., LTD. by using BROOKFIELD programmable DV-II+Viscometer) of the chitin was changed into about 23,000 dalton.

(2) After the chitin from step (1) was dried, the chitin was reacted with sodium hydroxide (NaOH; 42.5 wt %) for deacetylation. The solid/liquid ratio (i.e. chitin/sodium hydroxide) was 1:45. The reaction was maintain under 40° C. Mixtures containing chitosan of 60% and 90% deacetylation were obtained respectively upon different reaction time (15 hours and 30 hours, respectively).

(3) Then, chitosan of 60% deacetylation and chitosan of 90% deacetylation were processed respectively as follows:
3-1 Chitosan of 60% deacetylation was pressed to separate NaOH therein. Then, the pressed chitosan of 60% deacetylation was neutralized with 10 wt % H₂SO₄ to form a solution containing water soluble 60% deacetylation chitosan. This solution was purified by dialysis (Spectrum Labs Hollow Fiber) to remove Na₂SO₄. Then citric acid (40 wt %) was added in to react with the chitosan. The reaction was conducted at 30° C. for 2 hours. After the reaction, the solution containing chitosan citrate of 60% deacetylation was obtained and Freeze-Dried and Grinded to provide powder thereof.
3-2 Chitosan of 90% deacetylation was pressed to separate NaOH therein. Then, the chitosan of 90% deacetylation was washed with water and neutralized with 10 wt % H₂SO₄. After that, the chitosan of 90% deacetylation was again pressed to separate from the liquid therein. Then citric acid (40 wt %) was added in to react with the chitosan. The reaction was conducted at 30° C. for 24 hours. After the reaction, chitosan citrate of 90% deacetylation was obtained and spray dried to provide powder thereof.

(4) The obtained chitosan citrates were formulated as aqueous solution of various concentrations for the subsequent phosphate-binding assay.

### Experiment 2: Phosphate Binding Assay

Since the phosphate uptake in human body mostly takes place in the intestine instead of stomach, we tested the phosphate-binding ability of the aforesaid 60% deacetylation chitosan citrate (CHS-60L) and 90% deacetylation chitosan citrate (CHS-90L) in an environment of pH 8.3, which mimicked to the intestine environment in vivo.

Briefly, KH₂PO₄ and the present chitosan citrate were mixed at room temperature (about 25° C.) for 2 hours. See Table 1. Then, 2 μl of the mixture of each group (and KH₂PO₄ without reaction with binding agents) was mixed with 120 μl of acetate buffer (0.1 N acetic acid and 0.025 N of CH₃COONa), 12 μl of ascorbic acid (1 wt %), and 12 ml of NH₄MoO₄ (1%, in 0.05 N H₂SO₄) at room temperature for 20 to 30 minutes. After that, the OD₇₀₀ of the mixture was detected by spectrometer. The binding ability was shown in the following Table 2. The conventional phosphate binding agent, calcium carbonate, was used as positive control of the experiment.

TABLE 1
Experiment Design.

| | |
|---|---|
| Group | Binding Agent |

| | | |
|---|---|---|
| Group 1 | KH₂PO₄ (0.53M) | CHS-60L (10 mM) |
| Group 2 | KH₂PO₄ (0.53M) | CHS-60L (20 mM) |
| Group 3 | KH₂PO₄ (0.53M) | CHS-90L (10 mM) |
| Group 4 | KH₂PO₄ (0.53M) | CHS-90L (20 mM) |
| Group 5 | KH₂PO₄ (0.53M) | calcium carbonate (10 mM) |
| Group 6 | KH₂PO₄ (0.53M) | calcium carbonate (20 mM) |

TABLE 2
Phosphate-binding ability

| Group | Binding Agent | Binding ability (%) |
|---|---|---|
| Group 1 | CHS-60L (10 mM) | 83.90/86.25 |
| Group 2 | CHS-60L (20 mM) | 94.60/94.95 |
| Group 3 | CHS-90L (10 mM) | 61.45/66.05 |
| Group 4 | CHS-90L (20 mM) | 53.05/42.30 |
| Group 5 | calcium carbonate (10 mM) | 70.90 |
| Group 6 | calcium carbonate (20 mM) | 75.40 |

*The phosphate binding ability was calculated by the formula: (OD₇₀₀ of mixture after binding reaction)/(OD₇₀₀ of KH₂PO₄ without reaction with binding agents) *100%.

The experiment result showed that CHS-60L exhibited better binding ability than CHS-90L as well as the conventional binding agent (calcium carbonate). The result confirmed that the common knowledge of the more deacetylation the better the binding ability is a bias as it failed to consider where does the phosphate update take place in human body.

What is claimed is:

1. A method for phosphate-binding comprising administering a subject in need an effective amount of an enteric coated pharmaceutical composition comprising chitosan citrate and a pharmaceutically acceptable carrier; wherein said chitosan citrate comprises a chitosan moiety of 50 to 75% deacetylation.

2. The method of claim 1, wherein said chitosan moiety is of 55 to 70% deacetylation.

3. The method of claim 1, wherein said pharmaceutically acceptable carrier comprises water, PBS, salt solutions, gelatins, oils, alcohols, glycerol, chitosan, alginate, chondroitin, Vitamin E, dimethyl sulfoxide (DMSO), or a combination thereof.

4. The method of claim 1, wherein said subject in need is a hyperphosphatemia patient.

5. The method of claim 1, wherein said enteric coated pharmaceutical composition is a tablet, a capsule or a microcapsule.

6. The method of claim 1, wherein said administering is through oral route.

7. The method of claim 1, wherein said administering is conducted before, simultaneously, or after diet of said subject in need.

8. The method of claim 1, wherein said enteric coated pharmaceutical composition comprises an enteric coating of methacrylic acid and acid ester polymers, polyvinyl acetate phthalate, cellacephate, cellulose acetate trimellitrate, carboxymethyl ethylcellulose, hydroxypropyl methylcellulose phthalate, hydroxypropyl methylcellulose acetate succinate, or a combination thereof.

9. The method of claim 1, wherein said enteric coated pharmaceutical composition further comprise a pharmaceutically acceptable excipient.

10. The method of claim 9, wherein said excipient comprises a disintegrating agent, a binder, a lubricant, a preservative, or a combination thereof.

11. An enteric coated pharmaceutical composition, comprising chitosan citrate,
a pharmaceutically acceptable carrier, and
an enteric coating;
wherein said chitosan citrate is coated by said enteric coating; wherein said chitosan citrate comprises a chitosan moiety of 50 to 75% deacetylation.

12. The enteric coated pharmaceutical composition of claim 11, wherein said chitosan moiety is of 55 to 70% deacetylation.

13. The enteric coated pharmaceutical composition of claim 11, wherein said pharmaceutically acceptable carrier comprises water, PBS, salt solutions, gelatins, oils, alcohols, glycerol, chitosan, alginate, chondroitin, Vitamin E, dimethyl sulfoxide (DMSO), or a combination thereof.

14. The enteric coated pharmaceutical composition of claim 11, wherein said enteric coating comprises methacrylic acid and acid ester polymers, polyvinyl acetate phthalate, cellacephate, cellulose acetate trimellitrate, carboxymethyl ethylcellulose, hydroxypropyl methylcellulose phthalate, hydroxypropyl methylcellulose acetate succinate, or a combination thereof.

15. The enteric coated pharmaceutical composition of claim 11, wherein said enteric coated pharmaceutical composition further comprise a pharmaceutically acceptable excipient.

16. The enteric coated pharmaceutical composition of claim 15, wherein said excipient comprises a disintegrating agent, a binder, a lubricant, a preservative, or a combination thereof.
